(19) United States
(12) Patent Application Publication
(10) Pub. No.: US 2009/0082845 A1
(43) Pub. Date: Mar. 26, 2009

(54) ALIGNMENT STENT APPARATUS AND METHOD

(75) Inventor: Michael V. Chobotov, Santa Rosa, CA (US)

Correspondence Address: GRANT ANDERSON LLP, GRANT ANDERSON LLP C/O PORTFOLIOIP, P.O. BOX 52050, MINNEAPOLIS, MN, 55402, US

(73) Assignee: BOSTON SCIENTIFIC CORPORATION, Maple Grove, MN (US)

(21) Appl. No.: 11/861,746

(22) Filed: Sep. 26, 2007

(51) Int. Cl. A61F 2/06

(52) U.S. Cl. 623/1.13

(57) ABSTRACT

The invention provides a stent-graft system comprising a graft member and a stent having a connection end interconnected with the graft member and a free end opposed thereto. A belt retaining structure is provided at the stent free end. A belt is releasably retained in the belt retaining structure and is configured to constrain the stent free end independent of the stent connection end. A method of securing at least one end of a stent-graft within a vessel is also provided.

### BACKGROUND OF THE INVENTION

The present invention relates to a system for the treatment of disorders of the vasculature. More specifically, the invention relates to a system for the treatment of disease or injury that potentially compromises the integrity of a flow conduit in the body. For example, an embodiment of the invention is useful in treating indications in the digestive and reproductive systems as well as indications in the cardiovascular system, including thoracic and abdominal aortic aneurysms, arterial dissections (such as those caused by traumatic injury), etc. that include a curved lumen.

Medical devices for placement in a human or other animal body are well known in the art. One class of medical devices comprises endoluminal devices such as stents, stent-grafts, filters, coils, occlusion baskets, valves, and the like. A stent typically is an elongated device used to support an intraluminal wall. In the case of a stenosis, for example, a stent provides an unobstructed conduit through a body lumen is in the area of the stenosis. Such a stent may also have a prosthetic graft layer of fabric or covering lining the inside and/or outside thereof. A covered stent is commonly referred to in the art as an intraluminal prosthesis, an endoluminal or endovascular graft (EVG), a stent-graft, or endograft.

An endograft may be used, for example, to treat a vascular aneurysm by removing or reducing the pressure on a weakened part of an artery so as to reduce the risk of rupture. Typically, an endograft is implanted in a blood vessel at the site of a stenosis or aneurysm endoluminally, i.e. by so-called “minimally invasive techniques” in which the endograft, typically restrained in a radially compressed configuration by a sheath, crocheted or knit web, catheter or other means, is delivered by an endograft delivery system or “introducer” to the site where it is required. The introducer may enter the vessel or lumen from an access location outside the body, such as purcutaneously through the patient's skin, or by a “cut down” technique in which the entry vessel or lumen is exposed by minor surgical means. The term “proximal” as used herein refers to portions of the endograft, stent or delivery system relatively closer to the end outside of the body, whereas the term “distal” is used to refer to portions relatively closer to the end inside the body.

After the introducer is advanced into the body lumen to the endograft deployment location, the introducer is manipulated to cause the endograft to be deployed from its constrained configuration, whereupon the stent is expanded to a predetermined diameter at the deployment location, and the introducer is withdrawn. Stent expansion typically is effected by spring elasticity, balloon expansion, and/or by the self-expansion of a thermally or stress-induced return of a memory material to a pre-conditioned expanded configuration.

Among the many applications for endografts is that of deployment in lumen for repair of an aneurysm, such as a thorasic aortic aneurysm (TAA) or an abdominal aortic aneurysm (AAA). An AAA is an area of increased aortic diameter that generally extends from just below the renal arteries to the aortic bifurcation and a TAA most often occurs in the descending thoracic aorta. AAA and TAA generally result from deterioration of the arterial wall, causing a decrease in the structural and elastic properties of the artery. In addition to a loss of elasticity, this deterioration also causes a slow and continuous dilation of the lumen.

The standard surgical repair of AAA or TAA is an extensive and invasive procedure typically requiring a week long hospital stay and an extended recovery period. To avoid the complications of the surgical procedure, practitioners commonly resort to a minimally invasive procedure using an endoluminal endograft to reinforce the weakened vessel wall, as mentioned above. At the site of the aneurysm, the practitioner deploys the endograft, anchoring it above and below the aneurysm to relatively healthy tissue. The anchored endograft diverts blood flow away from the weakened arterial wall, minimizing the exposure of the aneurysm to high pressure.

Intraluminal stents for repairing a damaged or diseased artery or to be used in conjunction with a graft for delivery to an area of a body lumen that has been weakened by disease or damaged, such as an aneurysm of the thorasic or abdominal aorta, are well established in the art of medical science.

While intraluminal stents are advantageous in anchoring the device, an improved system for aligning stents in curved vessels or lumens is desired.

### SUMMARY OF THE INVENTION

In one aspect, the invention provides a stent-graft system comprising a graft member and a single segment stent having a connection end interconnected with the graft member and a free end opposed thereto. A belt retaining structure is provided at the stent free end. A belt is releasably retained in the belt retaining structure and is configured to constrain the stent free end independent of the stent connection end.

In another aspect, the invention provides a method of securing at least one end of a graft within a vessel. The method comprises: positioning within the vessel a stent-graft comprising a single segment stent and a graft with a connection 15 end of the stent connected to an end of the graft, the stent having a free end opposite the connection end, the stent free end including a belt retaining structure with a belt releasably retained thereabout; deploying the stent connection end within the vessel; repositioning the stent-graft within the vessel; and releasing the belt to deploy the free end of the stent.

Other aspects and advantages of the present invention will be apparent from the detailed description of the invention provided hereinafter.

### BRIEF DESCRIPTION OF THE DRAWINGS

The invention is best understood from the following detailed description when read in connection with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 shows a portion of an endovascular graft according to an embodiment of the present invention in a contracted state for delivery through a catheter.

FIG. 2 shows a flat pattern of an embodiment of a stent in accordance with the present invention.

FIG. 3 shows a portion of an endovascular graft according to an embodiment of the present invention partially deployed within an aortic arch of the patient.

FIG. 4 shows the endovascular graft portion of FIG. 3 fully deployed within the internal vasculature of the patient.

### DETAILED DESCRIPTION OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

Referring to FIG. 1, a portion of an illustrative endovascular graft 10 is shown in its contracted configuration. Unless otherwise stated, the term “graft” or “endovascular graft” is used herein to refer to a prosthesis capable of repairing and/or replacing diseased vessels or portions thereof, including generally tubular and bifurcated devices and any components attached or integral thereto. For purposes of illustration, the graft embodiments described herein may be used in the endovascular treatment of abdominal aortic aneurysms (AAA) or thoracic aortic aneurysms, however, other applications are within the scope of the present invention. For the purposes of this application, with reference to endovascular graft devices, the term “proximal” describes the end of the graft that will be oriented towards the oncoming flow of bodily fluid, typically blood, when the device is deployed within a body passageway. The term “distal” therefore describes the graft end opposite the proximal end. Finally, while the drawings in the various figures are accurate representations of the various embodiments of the present invention, the proportions of the various components thereof are not necessarily shown to exact scale within and among or between any given figure(s).

An end of the graft 10 is illustrated and may represent the proximal or distal end of the graft 10. The graft 10 includes a generally tubular structure or graft body section 13 comprised of one or more layers of fusible material, such as expanded polytetrafluoroethylene (ePTFE). An inflatable cuff 16 is disposed at or near the end 14 of graft body section 13. A neck portion 23 is disposed in the vicinity of graft body section end 14 and serves as an additional means to help seal the deployed graft against the inside of a body passageway. Graft body section 13 forms a longitudinal lumen 22 configured to confine a flow of fluid therethrough.

An attachment ring 24 is affixed to or integrally formed in graft body section 13, or as shown in FIG. 1, at or near graft body section end 14 and neck portion 23. In the embodiment of FIG. 1, attachment ring 24 is a serpentine ring structure comprising apices 28. Other embodiments of attachment ring 24 may take different configurations. Attachment ring 24 may be made from any suitable material that permits expansion from a constrained state, most usefully a shape memory alloy having superelastic properties such as nickel titanium (NiTi). Other suitable attachment ring 24 materials include stainless steel, nickel-cobalt alloys such as MP35N, tantalum and its alloys, polymeric materials, composites, and the like. Attachment ring 24 (as well as all stents and attachment rings described herein) may be configured to self-expand from the illustrated radially constrained state.

Some apices 28 may also comprise a attachment ring connector element not shown). The number of connector elements may vary and can be distributed, for example, on every apex, every third or fourth apex, or any other pattern are within the scope of the present invention.

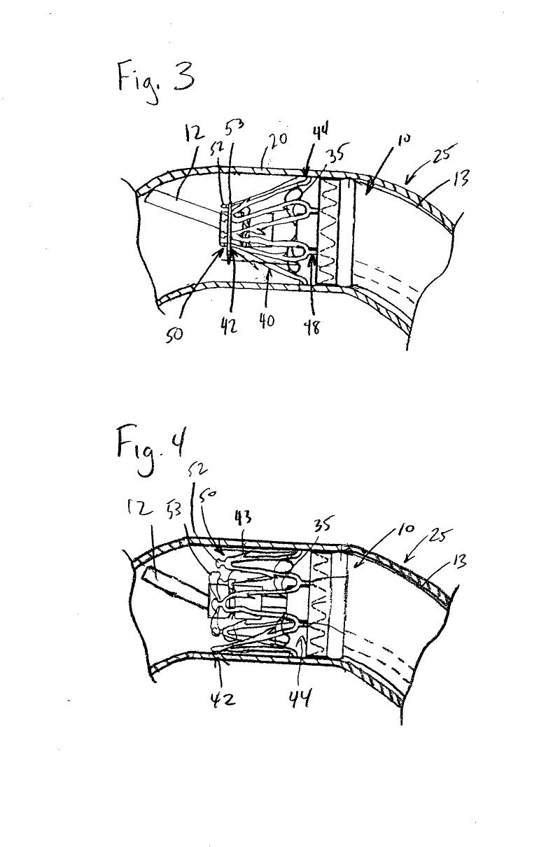

Graft 10 further comprises one or more stents 40 having, in the deployed state (see FIG. 4), a generally free end 42 and a connection end 44. FIGS. 1-4 illustrate a proximal stent 40, but the stents 40 may additionally or alternatively be provided on the distal end of the graft 10. In the present embodiment, the stent 40 is desirably for use in an angulated or curved lumen and is a short aspect ratio stent including a single segment. By single segment stent, it is meant that the stent 40 includes single lengths of struts 41 between the free end 42 and a connection end 44.

As shown in FIGS. 1-4, stent 40 is typically, though not necessarily, made a part of graft 10 by having the connection end 44 affixed or connected to attachment ring 24 via connector elements as described in detail below. The connection end 44 of stent 40 may also be affixed or embedded directly to or in neck portion 23 and/or other portions of graft body section 13. In addition, the attachment ring and the stent may not be mechanically or otherwise fastened to one another but rather unified, formed of a monolithic piece of material, such as NiTi.

This configuration of stent 40, attachment ring 24, neck portion 23, and cuff 16 helps to separate the sealing function of cuff 16, which requires conformation and apposition to the vessel wall within which graft 10 is deployed without excessive radial force, from the anchoring function of stent 40 (attachment ring 24 and neck portion 23 play intermediate roles). As will be described in more detail hereinafter, the stents 40 of the present invention permit improved positioning of the graft 10 prior to stent anchoring, thereby facilitating better placement and sealing of the graft 10.

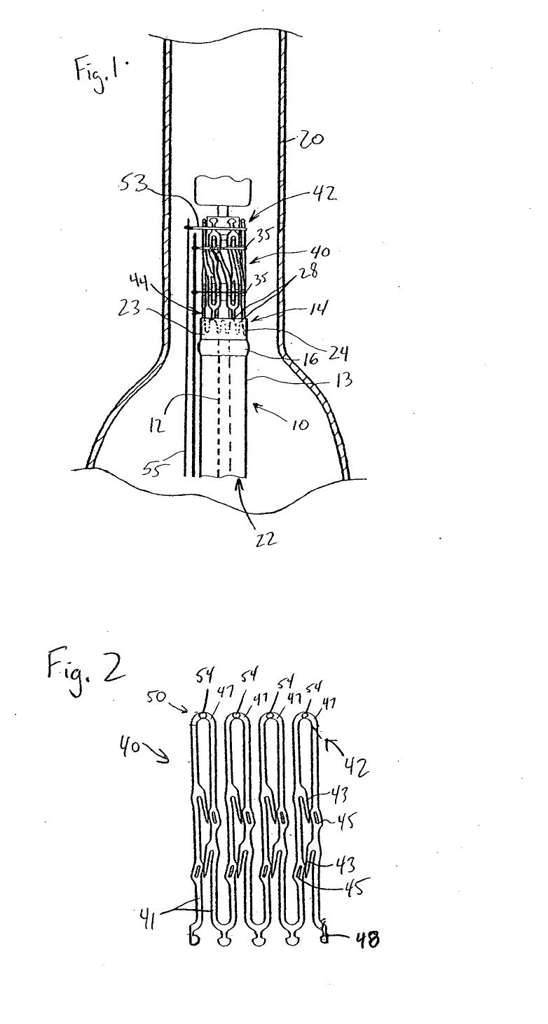

Referring to FIGS. 2-4, each stent 40 of the present invention generally comprises a series of interconnected struts 41. The struts 41 can have various configurations and lengths. Each stent 40 further comprises stent connector elements 48 at the connection end 44 thereof. The stent connector elements 48 are configured to be affixed or otherwise connected to attachment ring connector elements via coupling members (not shown), for example, threads or wires. The stents 40 may be manufactured from any suitable material, including the materials suitable for attachment ring 24. When manufactured from a shape memory alloy having superelastic properties such as NiTi, the stents 40 may be configured to self-expand upon release from the contracted state. The strut structure is often formed as a flat structure, as illustrated in FIG. 2, and thereafter, wrapped and connected in a cylindrical or other configuration, as illustrated in FIG. 1.

Each stent 40 includes one or more barbs 43. A barb 43 can be any outwardly directed protuberance, typically terminating in a sharp point that is capable of at least partially penetrating a body passageway in which graft 10 is deployed (typically the initial and medial layers of a blood vessel such as the abdominal aorta). The number of barbs, the length of each barb, each barb angle, and the barb orientation may vary from barb to barb within a single stent 40 or between multiple stents 40 within a single graft. Although the various barbs 43 (and tuck pads 45 discussed below) may be attached to or fixed on the stent struts 41, it is preferred that they be integrally formed as part of the stent struts 41, as shown in the various figures.

When stent 40 is deployed in the abdominal aorta, for example, typically in a location proximal to the aneurysm and any diseased tissue, barbs 43 are designed to work in conjunction with the distally-oriented blood flow field in this location to penetrate tissue and prevent axial migration of graft 10. As such, the barbs 43 in the FIG. 1 embodiment are oriented distally with respect to graft body section 13. However, the number, dimensions, configuration and orientation of barbs 43 may vary significantly, yet be within the scope of the present invention.

Struts 41 may also comprise optional integral tuck pads 45 disposed opposite each barb 43. During preparation of graft 10 (and therefore the stents 40) into its reduced diameter delivery configuration, each barb 43 is placed behind a corresponding strut 41 and/or optional tuck pad 45, if present, to thereby prevent the is barbs 43 from contacting the inside of a delivery sheath or catheter during delivery of the device and from undesired contact with the inside of a vessel wall. As described in U.S. Pat. No. 6,761,733 to Chobotov et al., the complete disclosure of which is incorporated herein by reference, an initial stage release belt 35 disposed about the struts 41 retain the stent 40 in this delivery configuration. The initial stage release belts 35 retain the contracted stent 40 on a guidewire chassis 12 or the like.

The number of initial stage belts 35 varies in accordance with the structure of the stent 40. The stent 40 as illustrated in FIGS. 1-4 is a single segment and includes only one initial stage belt 35 about the proximal end of the stent 40. The stent 40 of the present invention includes a belt retaining structure 50 provided along the crowns 47 at the free end 42 of the stent 40. In the embodiment illustrated in FIGS. 1 and 3-4, the belt retaining structure 50 includes a plurality of mushroom shaped connectors 52 extending from the crowns 47. The mushroom shaped connectors 52 may be provided at each crown 47, as illustrated, or in any configuration with respect to the crowns 47. Referring to FIGS. 1 and 5, a releasable secondary stage belt 53 is positionable about the mushroom shaped connectors 52 to retain the stent free end 42 in a contracted state until the secondary stage belt 53 is released, for example, via a release wire 55. In the embodiment illustrated in FIG. 2, the belt retaining structure 50 includes a through hole 54 provided in a plurality of the crowns 47. A releasable belt (not shown) is threaded through the through holes 54 and pulled tight to retain the stent free end 42 in a contracted state until the belt is released. Other belt retaining structures 50 along the stent free end 42 may also be utilized.

As shown in FIG. 3, upon release of the initial stage belts 35, the stent connection end 44, the attachment ring 24, and the graft 10 expand while the secondary stage belt 53 engages the belt retaining structure 50 and retains the stent free end 42 in the generally contracted condition. The stent connection end 44 and the graft 10 expand based on the self expanding nature of the stent 40 and also the force of the distal fluid flow into the graft 10. The struts 41 and barbs 43 are configured such that when the belt retaining structure 50 is in place and the stent free end 42 is restrained, the barbs 43 do not extend sufficiently radially to engage the vessel wall 20, but instead remain spaced therefrom. As such, the graft 10 and stent 40 may be moved and repositioned without the barbs 43 engaging and damaging the vessel wall 20. In at least one embodiment of the invention, the barbs 43 are axially positioned closer to the stent free end 42 than the stent connection end 44 to further ensure the barbs 43 will not contact the vessel wall 20 in the partially deployed state.

Once the stent 40 and graft 10 are positioned as desired, the release wire 55 may be pulled to release the secondary stage belt 53 from the belt retaining structure 50, thereby allowing the stent 40 to fully deploy as illustrated in FIG. 4. Upon full deployment, the struts 41 are free to fully radially expand such that the barbs 43 engage the vessel wall 20 in a normal manner.

In addition to facilitating manual movement and repositioning of the graft 10 and stent 40, the staged deployment of the stent 40 also facilitates self-alignment of the stent 40 and graft 10. As explained above, upon release of the initial stage belts 35, the graft 10 is free to expand and distal fluid flow flows into the graft 10 and creates a “windsock” effect. That is, the distal fluid flow expands the graft 10 and applies a slight distal force upon the graft 10. This distal force helps to align the graft 10 and the stent 40 within the vessel.

This self alignment is particularly advantageous during deployment of a stent graft within an angulated vessel, for example, in the aortic arch. Referring to FIG. 3, the stent 40 is illustrated partially deployed in an aortic arch 25. The delivery guidewire chassis 12 contacts the vessel wall 20 and does not remain coaxial with respect to the arch 25. As such, in the initial delivery position, the stent 40 may be cocked or otherwise misaligned with respect to the vessel wall 20. In a prior art single stage deployment, the stent would expand and the barbs would engage the vessel wall even if the stent was misaligned. With the stent 40 of the present invention, the initial stage belt(s) 35 are released and the stent 40 is partially deployed. The distal fluid flow flows into the graft 10 and creates the windsock effect, thereby pulling the graft 10 and stent 40 into alignment with the flow and thereby the vessel wall 20.

While preferred embodiments of the invention have been shown and described herein, it will be understood that such embodiments are provided by way of example only. Numerous variations, changes and substitutions will occur to those skilled in the art without departing from the spirit of the invention. Accordingly, it is intended that the appended claims cover all such variations as fall within the spirit and scope of the invention.

What is claimed:

1. A stent-graft system comprising:
a graft member;
a single segment stent having a connection end interconnected with the graft member and a free end opposed thereto;
a belt retaining structure provided at the stent free end; and
a belt releasably engaging the belt retaining structure and configured to constrain the stent free end substantially independent of the stent connection end.

2. The stent-graft system according to claim 1 wherein the stent comprises a plurality of struts extending between the connection end and the free end.

3. The stent-graft system according to claim 2 wherein crowns adjoin respective adjacent struts at the free end of the stent.

4. The stent-graft system according to claim 3 wherein the belt retaining structure includes a plurality of through holes extending through the crowns.

5. The stent-graft system according to claim 4 wherein the belt is threaded through a plurality of the through holes.

6. The stent-graft system according to claim 3 wherein the belt retaining structure includes at least two mushroom shaped connectors extending from respective crowns.

7. The stent-graft system according to claim 2 wherein one or more barbs extend from the stent struts.

8. The stent-graft system according to claim 7 wherein the barbs are positioned closer to the stent free end than the stent connection end.

9. The stent-graft system according to claim 1 wherein the belt is releasably secured by a release wire.

10. The stent-graft system according to claim 1 wherein at least one additional belt is releasably secured about the stent between the connection end and the free end and is releasable independent from the belt.

11. The stent-graft system according to claim 1 wherein the stent connection end includes a plurality of connection elements configured for attachment to corresponding connection members on the tubular graft.

12. A method of securing at least one end of a stent-graft within a vessel, comprising:
positioning within the vessel a stent-graft comprising a single segment stent and a graft with a connection end of the stent connected to an end of the graft, 5 the stent having a free end opposite the connection end, the stent free end including a belt retaining structure with a belt releasably retained thereabout;
deploying the stent connection end within the vessel;
repositioning the stent-graft within the vessel, if needed; and
releasing the belt to deploy the free end of the stent.

13. The method according to claim 12 wherein the vessel is a thoracic aorta.

14. The method according to claim 12 wherein the vessel is an abdominal aorta.

15. The method according to claim 12 wherein the step of repositioning the stent-graft within the vessel includes moving the stent, the graft or a combination of the stent and the graft.

16. The method according to claim 12 wherein the step of repositioning the stent-graft within the vessel includes allowing a fluid flow through the vessel to enter within the graft to self-align the stent and graft.

17. The method according to claim 12 wherein the step of deploying the stent connection end within the vessel includes releasing an additional belt constraining the stent connection end.
